(19) United States
(12) Patent Application Publication
(10) Pub. No.: US 2025/0318892 A1
(43) Pub. Date: Oct. 16, 2025

(54) CORD AND DEVICE MANAGEMENT SYSTEM

(71) Applicant: Jamie Michelle Womack, Weaverville, NC (US)

(72) Inventor: Jamie Michelle Womack, Weaverville, NC (US)

(21) Appl. No.: 18/632,748

(22) Filed: Apr. 11, 2024

(51) Int. Cl. A61B 50/20; A61B 46/23; A61B 50/15; A61M 25/02

(52) U.S. Cl. CPC A61B 50/20; A61B 46/23; A61B 50/15; A61M 2025/028

(57) ABSTRACT

A cord and device management system includes a block and a removable section. The block defines a cavity therethrough, and the cavity is configured to accommodate at least one of a surgical device or a cord. A first side of the one or more sides of the block includes a curved portion. The removable section is configured to selectively connect to the curved portion of the block. The removable section includes a first side configured to at least partially conform to the contour of the curved portion of the block and a second side configured to, when the removable section is connected to the curved portion of the block, align with the first side of the block.

### TECHNICAL FIELD

This disclosure relates generally to a cord and device management system, in particular, to a cord and device management system for use in minimally invasive surgeries.

### BACKGROUND

Minimally invasive surgeries (MIS), such as orthopedic procedures, arthroscopic procedures, and laparoscopic procedures, are scoped surgeries which may use multiple corded power devices to provide, e.g., illumination, visualization, hemostasis, debridement, and/or suction inside a joint or body cavity. Consequently, the large number of cords used during procedures frequently causes devices to become tangled, slowing down procedure progress. Additionally, lack of positive control of corded devices can result in a corded device falling out of the sterile field, thereby requiring the device to be replaced. The motion of a cord being pulled back and forth at the border of the sterile field can also compromise the sterile field. Some procedures, for example, a total shoulder replacement, require an additional worker chiefly to manage the corded devices.

### SUMMARY

An aspect of this disclosure provides a cord and device management system. The cord and device management system includes a block and a removable section. The block defines a first cavity therethrough. The first cavity is configured to accommodate at least one of a device or a cord. The block includes one or more sides, and a first side of the one or more sides includes a curved portion. The removable section is configured to selectively connect to the curved portion of the block. The removable section includes a first side configured to at least partially conform to the curved portion of the block and a second side configured to, when the removable section is connected to the curved portion of the block, align with the first side of the block.

In another aspect of this disclosure, the device may be a surgical device.

In yet another aspect of this disclosure, the system may further include an insert configured to be disposed within at least one of the curved portion or the first cavity of the block. The insert may define an aperture, and the aperture may be configured to accommodate at least one of a device or a cord.

In a further aspect of this disclosure, the aperture may be open at each end of the insert to permit passage of at least one of the device or the cord through the insert.

In an aspect of this disclosure, the insert may be nonflammable.

In another aspect of this disclosure, the block may be disposable.

In yet another aspect of this disclosure, the block may be constructed from foam.

In a further aspect of this disclosure, the curved portion may be configured to conform to a contour of a portion of a body of a patient.

In an aspect of this disclosure, the block may include adhesive configured to connect the block to at least one of a surgical drape or a surgical instrument table.

In another aspect of this disclosure, the adhesive may be disposed on the curved portion of the block.

In yet another aspect of this disclosure, the system may further include one or more straps configured to bundle the cord together.

In a further aspect of this disclosure, the block may include a second side, a third side, and a fourth side. The block may be configured to stand upright on a flat surface on at least one of the first side, the second side, the third side, or the fourth side.

In an aspect of this disclosure, the first cavity may be disposed adjacent to the third side.

In another aspect of this disclosure, the system may further include a second cavity and a third cavity each configured to accommodate at least one of a device or a cord.

In yet another aspect of this disclosure, the system may further include a second insert configured to be disposed within the second cavity of the block and a third insert configured to be disposed within the third cavity of the block. Each of the second insert and the third insert may define an aperture configured to permit passage of at least one of a device or a cord through each of the second insert and the third insert.

In a further aspect of this disclosure, the third cavity may be disposed adjacent to the fourth side.

In another aspect of this disclosure, the second cavity may be approximately centered between the third side and the fourth side of the block.

In yet another aspect of this disclosure, the second cavity may be relatively larger than each of the first cavity and the third cavity.

In a further aspect of this disclosure, the block may be configured for use with a surgical instrument table.

In an aspect of this disclosure, the curved portion may be configured to support at least one of a device or a cord.

In another aspect of this disclosure, at least one of the first cavity or the curved portion may be configured to store a nonflammable container.

Further details and aspects of the present disclosure are described in more detail below with reference to the appended drawings.

### BRIEF DESCRIPTION OF DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1A illustrates an exemplary cord and device management system, in accordance with aspects of this disclosure;

FIG. 1B shows a removable section and a curved portion of the cord and device management system of FIG. 1A, in accordance with aspects of this disclosure;

FIG. 2 depicts the cord and device management system of FIG. 1A supporting surgical devices, with the removable section omitted and a surgical device supported within the curved portion, in accordance with aspects of this disclosure; and

FIG. 3 illustrates the cord and device management system of FIG. 1A as used during surgery, with the cord and device management system supported atop a surgical stand, in accordance with aspects of this disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

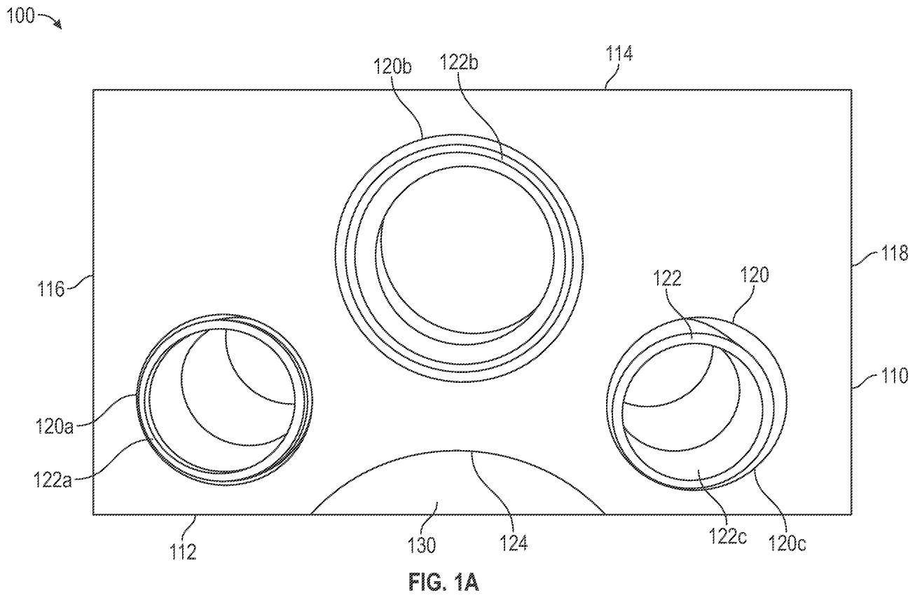

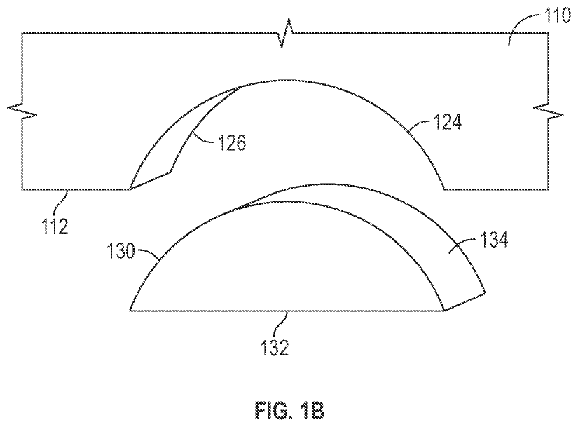

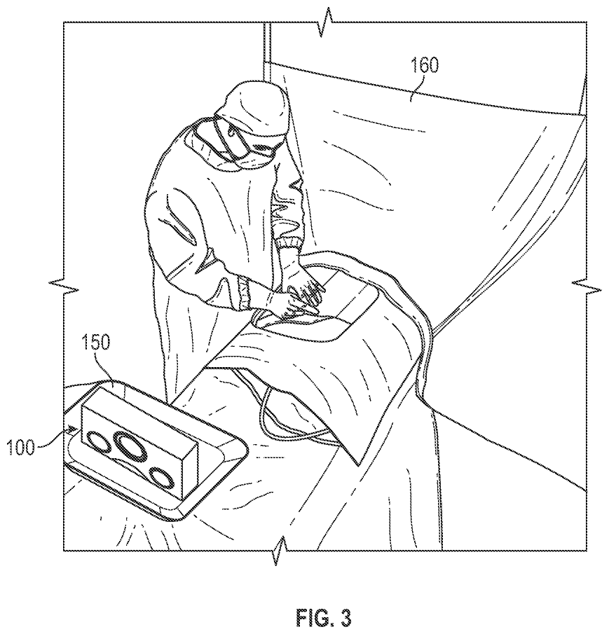

Referring to FIGS. 1A and 1B, a cord and device management system 100 for organizing cords and devices during surgery is shown. System 100 generally includes a block 110 and a removable section 130. System 100 is sterile for safe use during surgery and may be disposable for ease of use and to ensure sterility. Accordingly, block 110, removable section 130, or both may be manufactured from foam, such as a high-density foam, or from another material of a suitable density to support one or more surgical devices. For example, block 110 and removable section 130 may be manufactured from an open cell foam of weight 1.5/42 or 1.2/42. Block 110 may be rectangular, and may include a bottom side 112, a top side 114, a left side 116, and a right side 118. For example, block 110 may be approximately seven inches thick, and may include a bottom side 112 and a top side 114 of approximately fourteen inches wide and a left side 116 and a right side 118 of approximately nine inches tall. Alternatively, block 110 may be any suitable shape for holding cords and devices. Block 110 may be freestanding and may be stood upright on any of bottom side 112, top side 114, left side 116, or right side 118. In particular, system 100 may be suited for use atop a surgical instrument table capable of proximity to a patient, for example, a Mayo stand (see FIG. 3).

Block 110 additionally includes a curved portion 124, disposed along at least one of bottom side 112, top side 114, left side 116, and right side 118. As shown, curved portion 124 may define a cut out area along bottom side 112. In aspects, curved portion may be approximately one to one-and-a-quarter inches tall. Curved portion 124 may be horizontally centered on bottom side 112. For example, curved portion may be the top one-and-a-quarter inches of a circle which is twenty inches in diameter and centered on bottom side 112. Curved portion 124 may be shaped to conform to a contour of a portion of a patient's body such that system 100 may be supported on the patient's body during surgery. To further facilitate attachment of system 100 to a patient, curved portion 124 may include an adhesive 126, such as double-sided tape, glue, or another suitable adhesive, disposed thereon. Adhesive 126 may attach system 100 to a surgical drape 160 covering a patient, as will be described in greater detail regarding FIG. 3. Adhesive 126 may attach system 100 to a surgical instrument table 150 (see FIG. 3). Adhesive 126 may be applied to other portions of block 110, for example, bottom side 112. While portion 124 is described as curved, portion 124 may instead be any shape conducive to supporting block 110 atop a surface or object. For example, portion 124 may be a rectangular cut out, and may be inserted onto a rectangular object or an object including a rectangular protrusion of the same shape. Portion 124 may be an angled or diagonal cut, and may be configured to sit atop an inclined surface. In aspects, block 110 may include multiple portions 124 of the same or different shapes.

Removable section 130 may include one or more sides, such as a bottom side 132 and a top side 134. Top side 134 of removable section 130 may at least partially align with and conform to a shape of curved portion 124 of block 110. For instance, top side 134 of removable section 130 may follow an outline of curved portion 124 of block 110 such that, when removable section 130 is coupled to block 110, top side 134 is entirely flush against curved portion 124. In aspects, top side 134 may only contact parts of curved portion 124, leaving gaps between points of contact of top side 134 and curved portion 124. When removable section 130 is connected to block 110, bottom side 132 of removable section 130 may align with a side of block 110. For example, bottom side 132 of removable section 130 may align with bottom side 112 of block 110, as shown, such that bottom side 132 of removable section 130 does not protrude beyond or recess below bottom side 112 of block 110. Therefore, when removable section 130 is connected to a side of block 110, block 110 may rest stably on that side on a flat surface.

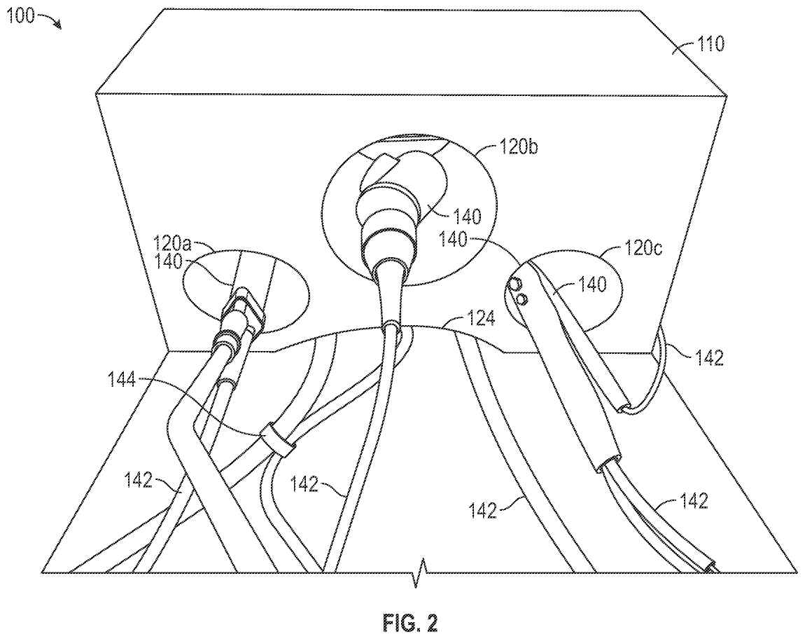

Block 110 additionally may include one or more cavities 120a-c configured to hold and support a surgical device 140 or a cord 142 thereof (FIG. 2). Cavities 120a-c may be circular or may be any shape suitable for storing a surgical device 140 or cord 142. Cavities 120a-c may be disposed entirely through block 110, such that a surgical device 140 or cord 142 may pass through block 110. In aspects, block 110 may be sufficiently rigid to support the weight of items within cavities 120a-c. In further aspects, an insert 122a-c, such as a tubular insert, may be inserted into each cavity 120a-c to provide structure to system 100. Each insert 122a-c may include an aperture therethrough, and each insert 122a-c may be dimensioned to accommodate one or more cords 142 or devices 140. In aspects, inserts 122a-c may be manufactured from PVC, cardboard, or another sufficiently rigid material. Each cavity 120a-c or corresponding insert 122a-c may be dimensioned to hold a particular surgical instrument, for example, an endoscope. The number of cavities 120 and corresponding inserts 122 included in system 100 may depend on the particular surgical procedure performed. As shown, system 100 may include a first cavity 120a and first insert 122a, a second cavity 120b and second insert 122b, and a third cavity 120c and third insert 122c, though the number of cavities 120 and inserts 122 may be greater or fewer as needed.

Referring to FIG. 2, system 100 is shown with surgical devices 140 and cords 142 held within cavities 120a-c and inserts 122a-c. Certain cavities 120 or inserts 122 may be designated for holding either a surgical instrument 140 or a cord 142 of the surgical instrument 140. Certain cavities 120 or inserts 122 may additionally be differently sized to accommodate larger surgical instruments 140 or a higher number of cords 142. In aspects, first cavity 120a and third cavity 120c may be configured to hold surgical instruments 140, while second cavity 120b may be configured to contain all cords 142 of surgical instruments 140. Accordingly, second cavity 120b may be relatively larger than first cavity 120a and third cavity 120c. For example, first cavity 120a and third cavity 120c may have a diameter of three inches, while second cavity 120b may have a diameter of four inches.

In certain embodiments, second cavity 120b may be centrally located on block 110 to allow for cords 142 of surgical instruments 140 to more conveniently reach second cavity 120b. For example, if bottom side 112 and top side 114 are each approximately fourteen inches wide and left side 116 and right side 118 are each approximately nine inches tall, a center point of second cavity 120b may be approximately seven inches to the right of left side 116 and approximately four-and-a-half inches down from top side 114. First cavity 120a may be disposed near left side 116 of block 110, for instance, first cavity 120a may be disposed approximately one-and-a-quarter inch right from left side 116 and down from top side 114. Third cavity 120c may be disposed near right side 118 of block 110, for example, third cavity 120c may be disposed approximately one-and-a-quarter inch left from right side 118 and down from top side 114. Second cavity 120b may be roughly centered between left side 116 and right side 118 of block 110. In the aforementioned configuration, surgical instruments 140 disposed in first cavity 120a and third cavity 120c are located approximately equidistant from second cavity 120b, and the cords 142 of surgical instruments 140 may be stored within second cavity 120b without unnecessary tension or pulling. Alternatively, or additionally, if block 110 rests on bottom side 112 on a flat surface, removable section 130 may be removed such that cords 142 may pass underneath curved portion 124 of block 110. To further aid in organizing cords 142, system 100 may include one or more straps 144 for bundling cords 142 of surgical instruments 140. As shown, a cord 142 of a surgical instrument 140 may be individually bundled using a strap 144, or a strap 144 may be used to consolidate all multiple cords 142 into a single bundle. The straps may include clips, hook and loop closures, or the like.

In aspects, as opposed to block 110 resting on bottom side 112 on a flat surface or curved portion 124 resting on a patient, block 110 may instead be inverted such that curved portion 124 faces upwards to support a surgical device 140 or cord 142. For example, curved portion 124 may support a surgical device which may be too large to fit within any of cavities 120a-c or inserts 122a-c. Curved portion 124 may also be used to support heat-generating surgical devices 140, such as cameras and cautery devices, to remove the heat-generating surgical devices 140 from reach of flammable items such as surgical drape 160. Nonflammable containers may also be rested atop any side of block 110, atop curved portion 124, or within cavities 120a-c or inserts 122a-c, in order to store heat-generating surgical instruments 140. In aspects, certain inserts 122 may themselves be nonflammable. Inserts 122 may also be configured to sit within curved portion 124. As stated earlier, inserts 122a-c may also be shaped and dimensioned for particular surgical instruments 140, and depending upon the surgical instrument 140 received within, may be configured as a pocket with a single opening rather than an aperture open at a first end and a second end. For example, an insert 122 including only an opening at the first end may be desirable to store a heat-generating surgical instrument 140, as the heat-generating surgical instrument 140 would be prevented from inadvertently falling through the insert 122 at the second end. Other configurations of inserts 122 are contemplated, for example inserts 122 may be trays, containers including closures, and the like.

FIG. 3 shows system 100 as used in a surgical procedure. As mentioned earlier, system 100 may be placed atop a surgical instrument table 150, such as a Mayo stand, or any table suitable for holding system 100 or bringing system 100 near a patient. Surgical instruments 140 which are corded have an operating range limited by a length of cord 142, therefore system 100 may be located as close to an operating site of a patient's body as possible. Locating surgical instrument table 150 in proximity to a patient ensures that manipulation of corded surgical instruments 140 does not pull system 100 out of a sterile field or pull surgical instruments 140 out of storage within system 100. Surgical instrument table 150 may be a single-legged table, allowing surgical instrument table 150 to be placed directly adjacent to or directly over a patient. Surgical instrument table 150 may include wheels (not shown) to provide ease of transportation.

In certain situations, system 100 may be required to be in closer proximity to a patient than may be provided by surgical instrument table 150. In these situations, curved portion 124 of system 100 may rest securely on a patient's body or on a surgical drape covering a patient's body. System 100 may be attached to surgical drape 160 using adhesive 126 as applied to curved portion 124 of block 110. Adhesive 126 may instead be applied to another portion of block 110 to attach system 100 to surgical drape 160. As previously noted, whether system 100 is supported by surgical instrument table 150 or surgical drape 160, system 100 may additionally include straps 144 which may be affixed to surgical drape 160 to prevent unintentional movement of surgical instruments 140 or the cords 142 thereof. Straps 144 may fix cords 142 to surgical instrument table 150 or block 110 as well.

Certain aspects of the present disclosure may include some, all, or none of the above advantages and/or one or more other advantages readily apparent to those skilled in the art from the drawings, descriptions, and claims included herein. Moreover, while specific advantages have been enumerated above, the various aspects of the present disclosure may include all, some, or none of the enumerated advantages and/or other advantages not specifically enumerated above.

The aspects disclosed herein are examples of the disclosure and may be embodied in various forms. For instance, although certain aspects herein are described as separate aspects, each of the aspects herein may be combined with one or more of the other aspects herein.

Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an aspect,” “in aspects,” “in various aspects,” “in some aspects,” or “in other aspects” may each refer to one or more of the same or different example Aspects provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The aspects described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A cord and device management system, comprising:
a block defining a first cavity therethrough, the first cavity configured to accommodate at least one of a device or a cord, the block including one or more sides, wherein a first side of the one or more sides includes a curved portion; and
a removable section configured to selectively connect to the curved portion of the block, the removable section including a first side configured to at least partially conform to the curved portion of the block and a second side configured to, when the removable section is connected to the curved portion of the block, align with the first side of the block.

2. The cord and device management system of claim 1, wherein the first cavity accommodates a surgical device.

3. The cord and device management system of claim 1, further comprising an insert configured to be disposed within at least one of the curved portion or the first cavity of the block, the insert defining an aperture configured to accommodate at least one of a device or a cord.

4. The cord and device management system of claim 3, wherein the aperture is open at each end of the insert to permit passage of at least one of a device or cord through the insert.

5. The cord and device system of claim 3, wherein the insert is nonflammable.

6. The cord and device management system of claim 1, wherein the block is disposable.

7. The cord and device management system of claim 1, wherein the block is constructed from foam.

8. The cord and device management system of claim 1, wherein the curved portion is configured to conform to a contour of a portion of a body of a patient.

9. The cord and device management system of claim 1, wherein the block includes adhesive configured to connect the block to at least one of a surgical drape or a surgical instrument table.

10. The cord and device management system of claim 9, wherein the adhesive is disposed on the curved portion of the block.

11. The cord and device management system of claim 1, further comprising one or more straps configured to bundle the cord together.

12. The cord and device management system of claim 1, wherein the block includes a second side, a third side, and a fourth side, and wherein the block is configured to stand upright on a flat surface on at least one of the first side, the second side, the third side, or the fourth side.

13. The cord and device management system of claim 12 wherein the first cavity is disposed adjacent to the third side.

14. The cord and device management system of claim 12, further comprising a second cavity and a third cavity each configured to accommodate at least one of a device or a cord.

15. The cord and device management system of claim 14, further comprising a second insert configured to be disposed within the second cavity of the block and a third insert configured to be disposed within the third cavity of the block, wherein each of the second insert and the third insert define an aperture configured to permit passage of at least one of a device or a cord through each of the second insert and the third insert.

16. The cord and device management system of claim 14, wherein the third cavity is disposed adjacent to the fourth side.

17. The cord and device management system of claim 14, wherein the second cavity is approximately centered between the third side and the fourth side of the block.

18. The cord and device management system of claim 14, wherein the second cavity is larger in diameter than each of the first cavity and the third cavity.

19. The cord and device management system of claim 1, wherein the block is configured for use with a surgical instrument table.

20. The cord and device management system of claim 1, wherein the curved portion is configured to support at least one of a device or a cord.
